(19) United States
(12) Patent Application Publication
(10) Pub. No.: US 2024/0181397 A1
(43) Pub. Date: Jun. 6, 2024

(54) HIGH-THROUGHPUT MICROEMULSIFICATION MEMBRANE

(71) Applicant: ProLynx LLC, San Francisco, CA (US)

(72) Inventors: Jeffrey C. HENISE, San Francisco, CA (US); Brian YAO, Oakland, CA (US)

(73) Assignee: ProLynx LLC, San Francisco, CA (US)

(21) Appl. No.: 18/282,976

(22) PCT Filed: Mar. 18, 2022
(86) PCT No.: PCT/US2022/020959

§ 371 Date: Sep. 19, 2023

Related U.S. Application Data
(60) Provisional application No. 63/163,705, filed on Mar. 19, 2021.

(51) Int. Cl. B01D 63/06; B01D 69/04; B01D 71/52

(52) U.S. Cl. CPC B01D 63/06; B01D 69/04; B01D 71/5222; B01D 2325/38

(57) ABSTRACT

The present disclosure is related to high-throughput membranes for preparation of microdroplet emulsions and microparticle suspensions and apparatus and systems comprising the same. Also provided are methods of preparing microdroplet emulsions and microparticle suspensions.

### CROSS REFERENCE TO RELATED APPLICATION

This application claims priority to and the benefit of U.S. Provisional Application No. 63/163,705, filed on Mar. 19, 2021, the disclosure of which is incorporated herein by reference in its entirety.

### FIELD

The present disclosure is related to high-throughput membranes for preparation of microdroplet emulsions and microparticle suspensions and apparatus and systems comprising the same. Also provided are methods of preparing microdroplet emulsions and microparticle suspensions.

### BACKGROUND

Microparticulate forms of therapeutic agents such as microspheres, microparticles, and microcapsules offer numerous advantages for drug delivery, often providing improved tolerance, efficacy, and convenience. The preparation of such microparticulate forms is technically challenging, particularly with regard to control over particle size distribution. It is typically required that the particle size distribution within a microparticulate formulation be tightly controlled, as particle size influences several key properties of the drug—injectability through a narrow-gauge needle so as to minimize pain upon administration; tissue distribution after injection; uptake by macrophages and subsequent allergic responses; and potentially drug-release and polymer dissolution behavior for microparticulate forms comprising drugs trapped within or attached to a polymeric matrix.

Microparticulate forms of materials are often prepared by initial formation of an emulsion of a liquid form of the material, either as a solution or a melt, suspended in an immiscible solvent (a dispersion or continuous phase). Upon chemical or phase transformation of the liquid material, the emulsion becomes a suspension of microparticles in the immiscible phase that may be isolated by various physical techniques. Thus, the particle size distribution in the final microparticulate material is typically controlled by the emulsification process.

Microfluidic technologies have been used to produce microspheres of hydrogels for drug-delivery having extremely narrow distributions of particle sizes (see, for example, PCT Publication No. WO2019/152672), however the throughput capacity of such systems is inherently limited. Larger-scale emulsification technologies tend to provide higher throughput at the expense of a wider particle size distribution. While the particle size distribution may be refined through the use of various sizing techniques, for example by sieving, this results in an overall material loss in the process that may be financially burdensome. Recent developments in membrane technology have improved the efficiency of large-scale emulsification, for example through the use of cross-flow emulsification processes in which the liquid form of the material to be formed into microparticles is pushed through a membrane comprising micro-scale pores into a flowing solution of the dispersion phase. For water-in-oil emulsions, such crossflow-emulsification membranes are typically fabricated from metal or glass, are typically expensive to produce, and require coating with a hydrophobic agent to provide proper wetting with the oil phase similar to microfluidics chips. Such coatings are technically challenging to prepare with the needed tolerances and erode over the period of membrane use, limiting the lifetime of the membrane. Coating failure during an emulsification run often results in unusable material. There is therefore an unmet need for emulsification membranes that are inexpensive and do not require hydrophobic coatings prior to use.

### SUMMARY

The present disclosure provides emulsification membranes made of hydrophobic plastic that are inexpensive, easy to produce, and do not require hydrophobic coating. These membranes are useful in the preparation of microdroplet emulsions and microparticle suspensions having controlled particle size.

In one aspect, provided is a membrane comprising a plurality of pores and a surface made from a hydrophobic plastic.

In another aspect, provided is an apparatus comprising a tubular membrane disclosed herein and an outer chamber.

In another aspect, provided is a system comprising an apparatus disclosed herein.

In another aspect, provided is a method of generating an emulsion of microdroplets of a first liquid in a second liquid that is immiscible with the first liquid using an apparatus disclosed herein, the method comprising
flowing the first liquid into the outer chamber; and
flowing the second liquid through the tubular membrane, wherein
the first liquid passes through the pores of the membrane, thereby forming an emulsion of microdroplets of the first liquid in the second liquid.

### BRIEF DESCRIPTION OF THE DRAWINGS

FIG. 1 illustrates a membrane in accordance with some embodiments.

FIG. 2 illustrates an apparatus in accordance with some embodiments.

FIG. 3A illustrates an apparatus in accordance with some embodiments. FIG. 3B illustrates a cross-section view of the apparatus.

FIG. 4 illustrates a system in accordance with some embodiments.

FIG. 5 illustrates a system in accordance with some embodiments.

FIG. 6 shows micrographs of droplets produced from PEEK membranes with different parameters. (A) Droplets produced from the 1/32″ outer diameter and 0.020″ inside diameter tube at 33 mL/min continuous phase and 12 mL/min dispersed phase. (B) Droplets produced from the 1/16″ outer diameter and 0.020″ inside diameter tube at 33 mL/min continuous phase and 12 mL/min dispersed phase. (C) Droplets produced from the 1/32″ outer diameter and 0.020″ inside diameter tube at 6 mL/min continuous phase and 4 mL/min dispersed phase.

### DETAILED DESCRIPTION

In one aspect, provided is a membrane comprising a plurality of pores and a surface made from a hydrophobic plastic. In some embodiments, the membrane is made from a hydrophobic plastic. In some embodiments, the hydrophobic plastic is polyether ether ketone (PEEK).

The membrane may be flat or curved. In some embodiments, the membrane is flat. In some embodiments, the membrane is curved. In some embodiments, the membrane is tubular (e.g., a tube through which liquid may flow along the central axis).

The pores in the membrane may be generated in the plastic by any suitable technique, such as laser drilling. In some embodiments, the pores have a diameter between about 1 μm and about 100 μm, between about 1 μm and about 90 μm, between about 1 μm and about 80 μm, between about 1 μm and about 70 μm, between about 1 μm and about 60 μm, between about 1 μm and about 50 μm, between about 1 μm and about 40 μm, between about 1 μm and about 30 μm, between about 1 μm and about 20 μm, between about 1 and about 10 μm, between about 10 μm and about 100 μm, between about 10 μm and about 90 μm, between about 10 μm and about 80 μm, between about 10 μm and about 70 μm, between about 10 μm and about 60 μm, between about 10 μm and about 50 μm, between about 10 μm and about 40 μm, between about 10 μm and about 30 μm, between about 10 μm and about 20 μm, between about 20 μm and about 100 μm, between about 20 μm and about 90 μm, between about 20 μm and about 80 μm, between about 20 μm and about 70 μm, between about 20 μm and about 60 μm, between about 20 μm and about 50 μm, between about 20 μm and about 40 μm, between about 20 μm and about 30 μm, between about 30 μm and about 100 μm, between about 30 μm and about 90 μm, between about 30 μm and about 80 μm, between about 30 μm and about 70 μm, between about 30 μm and about 60 μm, between about 30 μm and about 50 μm, between about 30 μm and about 40 μm, between about 40 μm and about 100 μm, between about 40 μm and about 90 μm, between about 40 μm and about 80 μm, between about 40 μm and about 70 μm, between about 40 μm and about 60 μm, between about 40 μm and about 50 μm, between about 50 μm and about 100 μm, between about 50 μm and about 90 μm, between about 50 μm and about 80 μm, between about 50 μm and about 70 μm, or between about 50 μm and about 60 μm. In some embodiments, the pores have a diameter of about 1 μm, about 5 μm, about 10 μm, about 15 μm, about 20 μm, about 25 μm, about 30 μm, about 35 μm, about 40 μm, about 45 μm, about 50 μm, about 55 μm, about 60 μm, about 65 μm, about 70 μm, about 75 μm, about 80 μm, about 85 μm, about 90 μm, about 95 μm, or about 100 μm.

In some embodiments, the pores are in a patterned arrangement. The pores may be spaced so as to retain the structural integrity of the membrane under the pressures of the emulsification process. In some embodiments, the patterned arrangement comprises a plurality of rows, wherein each row comprises a plurality of pores. In some embodiments, the distance between centers of two adjacent pores in a row is between about 5-times and about 100-times, between about 5-times and about 90-times, between about 5-times and about 80-times, between about 5-times and about 70-times, between about 5-times and about 60-times, between about 5-times and about 50-times, between about 5-times and about 40-times, between about 5-times and about 30-times, between about 5-times and about 25-times, between about 5-times and about 20-times, between about 5-times and about 15-times, or between about 5-times and about 10-times of the pore diameter. In some embodiments, the distance between centers of two adjacent pores in a row is about 5-times, about 10-times, about 15-times, about 20-times, about 25-times, about 30-times, about 35-times, about 40-times, about 45-times, about 50-times, about 55-times, about 60-times, about 65-times, about 70-times, about 75-times, about 80-times, about 85-times, about 90-times, about 95-times, or about 100-times of the pore diameter.

In some embodiments, the rows are parallel to each other and the distance between two adjacent rows can be same or different from the distance between the centers of two adjacent pores in a row. In some embodiments, the distance between two adjacent rows is between about 5-times and about 100-times, between about 5-times and about 90-times, between about 5-times and about 80-times, between about 5-times and about 70-times, between about 5-times and about 60-times, between about 5-times and about 50-times, between about 5-times and about 40-times, between about 5-times and about 30-times, between about 5-times and about 25-times, between about 5-times and about 20-times, between about 5-times and about 15-times, or between about 5-times and about 10-times of the pore diameter. In some embodiments, the distance between two adjacent rows is about 5-times, about 10-times, about 15-times, about 20-times, about 25-times, about 30-times, about 35-times, about 40-times, about 45-times, about 50-times, about 55-times, about 60-times, about 65-times, about 70-times, about 75-times, about 80-times, about 85-times, about 90-times, about 95-times, or about 100-times of the pore diameter.

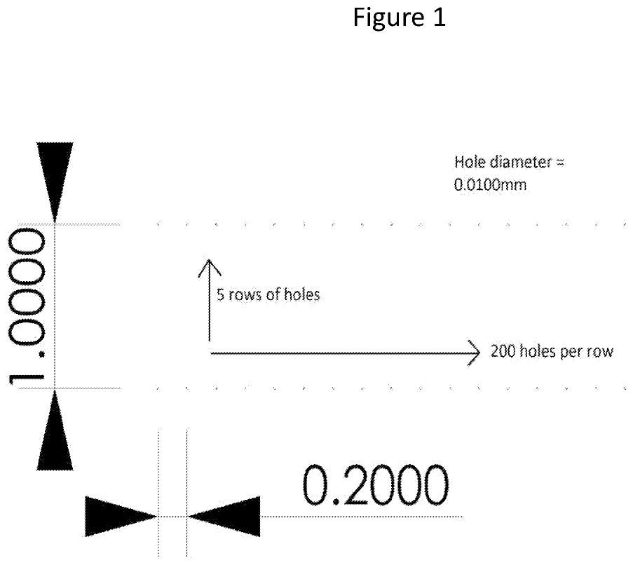

FIG. 1 illustrates a tubular membrane comprising a plurality of pores, in accordance with some embodiments. The pores in the membrane are arrayed in 5 rows of 200 pores where the rows are parallel to the tubular membrane's axis and spaced evenly around the tube's circumference. The tubular membrane may be created by laser drilling. In some embodiments, the tubular membrane is created from a PEEK tube, e.g., by laser drilling a PEEK tube. PEEK tubes are commercially available in diameters between 0.010″ (0.254 mm) and 1″ (25.4 mm) and wall thicknesses between 0.002″ (0.050 mm) to 0.010″ (0.254 mm), although other sizes may be custom manufactured. In some embodiments, the tubular PEEK membrane has an outside diameter of 1/16″ and an inside diameter of between 0.0025″ and 0.04″ (0.0635 to 1.016 mm). Such tubes are commercially available for use under pressure in liquid chromatography systems, and will support liquid flow rates up to 140 mL/min at a target velocity of 2.878 meters/sec. For the purpose of the present disclosure, PEEK has certain advantages: a) for water-in-oil emulsions, the hydrophobic material enables wetting by the continuous phase without surface treatment; b) it is chemically resistant to organic and aqueous media; c) it can be obtained in a USP Class VI compliant grade appropriate for pharmaceutical manufacturing; and d) it is highly temperature stable and can be sterilized in an autoclave so it is suitable for aseptic applications.

In another aspect, provided is an apparatus comprising a tubular membrane disclosed herein and an outer chamber. In some embodiments, the membrane may be placed inside the outer chamber. In some embodiments, the outer chamber is made from a metal such as stainless steel. In some embodiments, the outer chamber comprises an inlet through which a liquid can flow into the outer chamber. In some embodiments, the outer chamber comprises an inlet through which a liquid can flow into the outer chamber and an outlet through which the liquid can flow out of the outer chamber.

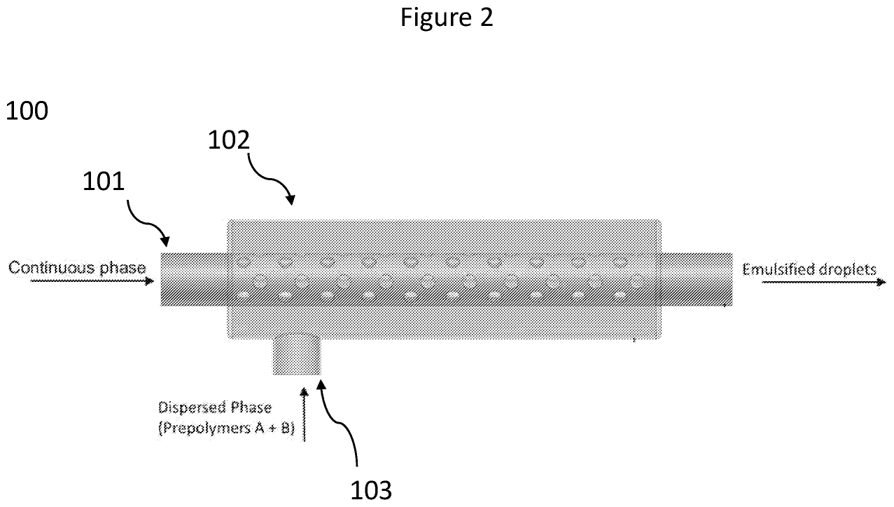

FIG. 2 illustrates an apparatus 100 comprising a membrane 101 and an outer chamber 102, in accordance with some embodiments. The outer chamber 102 has an inlet 103, through which a first liquid (dispersed phase) can flow into the outer chamber 102. A second liquid (continuous phase) flows through the membrane 101 along the central axis, thereby forming emulsified droplets.

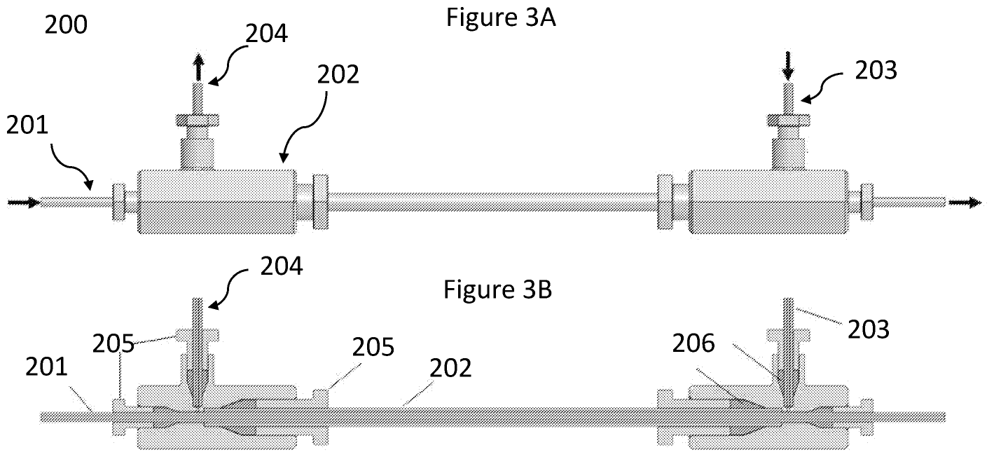

FIG. 3A illustrates an apparatus 200 comprising a membrane 201 and an outer chamber 202, in accordance with some embodiments. The outer chamber 202 has an inlet 203 and an outlet 204. The arrows indicate the flow of a first liquid (via inlet 203 and outlet 204) and the flow of a second liquid (through the tubular membrane 201 along the central axis). FIG. 3B illustrates a cross-section view of the apparatus 200. The membrane 201 is secured inside the outer chamber 202 with compression fitting nuts 205 and ferrules 206. The nuts and ferrules can have different sizes.

In another aspect, provided is a system comprising the membrane or the apparatus disclosed herein. In some embodiments, the system further comprises a means of flowing and pressurizing liquids into the outer chamber of the apparatus or through the tubular membrane, a means of measuring and controlling of the liquid flow rates and pressures, an overall process control device, and/or a means of collecting the microdroplet emulsion produced by the system. In some embodiments, the system further comprises filters for sterilization and/or removal of particulates that might clog the membrane pores. In some embodiments, the system is able to be sterilized for use in aseptic production of microparticulates, for example by autoclaving. In some embodiments, pumps or pressure may be used as a means of flowing and pressurizing liquids into the outer chamber of the apparatus or through the tubular membrane. In some embodiments, pulseless pumps of the type used for HPLC applications are used. In some embodiments, inert gas such as nitrogen or argon is used. In some embodiments, the use of inert gas pressure is preferred for dealing with volatile and flammable liquids. In some embodiments, liquid flow meters may be used to monitor and assist in the control the flow rates, and may be connected to the overall process control device (e.g., a computer) that provides feedback control of the flow rates. In some embodiments, the system may further comprise a container for collecting microdroplet emulsion generated by the system, a means of stirring the emulsion, a means of altering the temperature of the emulsion, and/or a means of refining the particle size distribution.

The membrane or the apparatus disclosed herein may also be used together with any previously-disclosed devices, for example those disclosed in PCT Patent Publication Nos. WO 2013/045918, WO 2014/006384, and WO 2019/092461.

In another aspect, provided herein is a method of generating an emulsion of microdroplets of a first liquid in a second liquid that is immiscible with the first liquid using an apparatus disclosed herein, the method comprising:
flowing the first liquid into an outer chamber; and
flowing the second liquid through a tubular membrane comprising a plurality of pores and a surface made from a hydrophobic plastic, wherein the tubular membrane is placed inside the outer chamber, wherein
the first liquid passes through the pores of the membrane, thereby forming an emulsion of microdroplets of the first liquid in the second liquid.

In some embodiments of a method disclosed herein, the first liquid is a buffered aqueous solution comprising equimolar amounts of two “prepolymers,” and the second liquid is a hydrocarbon containing surfactants. Examples of the first and second liquids that can be used include, without limitation, the liquids disclosed in U.S. Pat. Nos. 9,649,385 and 10,398,779; PCT Publication Nos. WO 2019/152672 and WO 2021/026494. In some embodiments, the first liquid is prepared immediately prior to introduction into the apparatus by mixing of separate streams of the two prepolymers. The mixture may also be formed prior to beginning the emulsification process depending on the rate of polymerization. The resulting suspension forms hydrogel microspheres after polymerization of the two prepolymers within the aqueous microdroplets. The resulting suspension of hydrogel microspheres may be subjected to a sieving process to refine the particle size distribution, and may further be sterilized by autoclaving as described in PCT Publication Nos. WO 2013/036847 and WO 2021/026494. In some embodiments, the hydrogels have the formula (I)

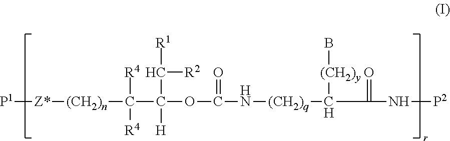

wherein P¹ and P² are each independently r-armed polyethylene glycols, wherein r=2-8;
Z* and B are connecting groups;
n=0-10;
R¹ and R² is each independently H, alkyl, or an electron-withdrawing group with the proviso that at least one of R¹ and R² is an electron-withdrawing group;
each R⁴ is independently C₁-C₃ alkyl, or taken together may form a 3-7 member ring; and
q and y are independently 0-6.

Electron-withdrawing groups are defined as groups having a Hammett sigma value greater than 0 (see, for example, Hansch et al. 1991 Chemical Reviews 91: 165-195). Typical examples are nitrile, nitro, sulfones, sulfoxides, carbonyls, and optionally substituted aromatics. In one embodiment, the electron-withdrawing groups are CN; NO₂; optionally substituted aryl or heteroaryl; SOₙR³ or COₙR³ wherein n=1-2 and R³=alkyl, aryl or heteroaryl, or N(R⁵)₂ or OR⁵ wherein each R⁵ is independently H, alkyl, aryl, or heteroaryl.

The emulsion of droplets may be collected in a holding/processing container for holding and further processing. Examples of such holding/processing container can be found in PCT Publication No. WO 2019/152672 and may comprise one or more of a means of stirring the emulsion, a means of altering the temperature of the emulsion, and a means of refining the particle size distribution.

In some embodiments of a method disclosed herein, the method further comprises converting the microdroplets to microparticles. In some embodiments, the method comprises collecting the microdroplets in a holding/processing container, wherein the microdroplets form a suspension of microparticles. In some embodiments, the method further comprises isolating and/or refining the microparticles. The microparticle suspension may be further refined by sizing, for example using sieves to removes particles that are too large or small. Such sieves may be placed in the bottom of the holding/processing container and are chosen such that they either retain or pass particles of a certain size distribution. In some embodiments, the initial microparticle suspension is first allowed to pass through a large-pore sieve chosen such that particles larger than the desired maximum diameter are retained on the sieve while particles of the desired size or smaller pass through into a second holding/processing container. This partially-refined microparticle suspension is then washed using a small-pore sieve chosen such that particles smaller than the desired minimum diameter pass through in the wash while particle of the desired size are retained in the holding/processing container. The sieves may be made of any suitable material having the desired pore sizes. In some embodiments, the sieves are made of woven steel mesh such as Dutch weave steel mesh.

In some embodiments, the microparticles are substantially uniform. In some embodiments, the microparticles are substantially uniform and have a size of about 10 μm, about 20 μm, about 30 μm, about 40 μm, about 50 μm, about 60 μm, about 70 μm, about 80 μm, about 90 μm, or about 100 μm.

In another aspect, provided herein is a microparticle or a microparticle suspension formed using method disclosed herein. In some embodiments, the microparticle is a microsphere of a poly(ethylene glycol) (PEG) hydrogel. Examples of hydrogels include, without limitation, the hydrogels disclosed in U.S. Pat. Nos. 9,649,385 and 10,398,779; PCT Publication Nos. WO 2019/152672 and WO 2021/026494.

The following examples illustrate, but do not limit the present disclosure.

### Preparation A. Prepolymers for Hydrogel Microsphere Formation

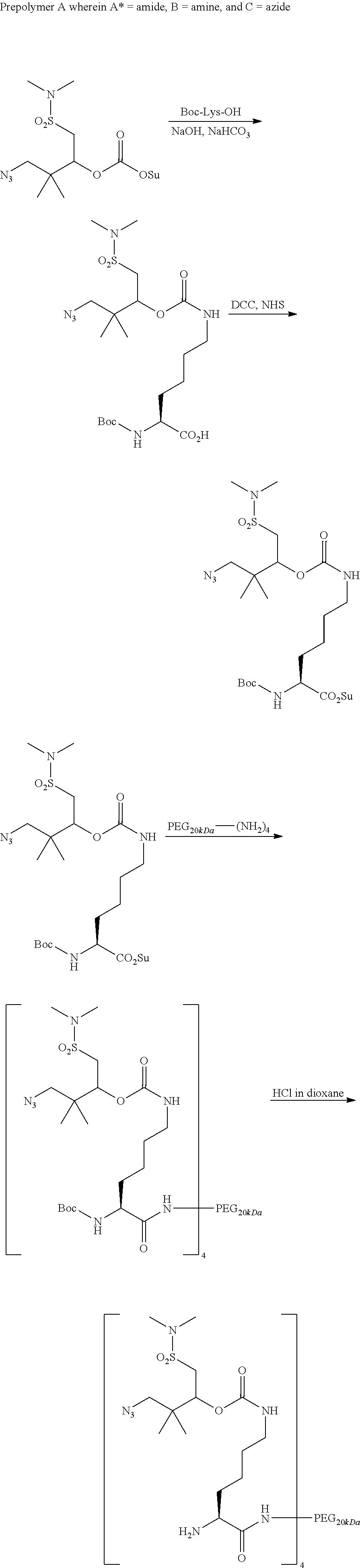

N^{α}-Boc-N^{ε}-{4-Azido-3,3-dimethyl-1-[(N,N-dimethyl)aminosulfonyl]-2-butyloxycarbonyl}-Lys-OH. A solution of Boc-Lys-OH (2.96 g, 12.0 mmol) in 28 mL of H₂O was successively treated with 1 M aq NaOH (12.0 mL, 12.0 mmol), 1 M aq NaHCO₃ (10.0 mL, 10.0 mmol), and a solution of O-{4-azido-3,3-dimethyl-1-[(N,N-dimethyl)aminosulfonyl]-2-butyl}-O′-succinimidyl carbonate (3.91 g, 10.0 mmol, 0.1 M final concentration) in 50 mL of MeCN. After stirring for 2 h at ambient temperature, the reaction was judged to be complete by C18 HPLC (ELSD). The reaction was quenched with 30 mL of 1 M KHSO₄ (aq). The mixture was partitioned between 500 mL of 1:1 EtOAc:H₂O. The aqueous phase was extracted with 100 mL of EtOAc. The combined organic phase was washed with H₂O and brine (100 mL each) then dried over MgSO₄, filtered, and concentrated by rotary evaporation to provide the crude title compound (5.22 g, 9.99 mmol, 99.9% crude yield) as a white foam. C18 HPLC, purity was determined by ELSD: 99.1% (RV=9.29 mL). LC-MS (m/z): calc, 521.2; obsd, 521.3 [M−H]⁻.

N^{α}-Boc-N^{ε}-{4-Azido-3,3-dimethyl-1-[(N,N-dimethyl)aminosulfonyl]-2-butyloxycarbonyl}-Lys-OSu. Dicyclohexylcarbodiimide (60% in xylenes, 2.6 M, 4.90 mL, 12.7 mmol) was added to a solution of N^{α}-Boc-N^{ε}-{4-azido-3,3-dimethyl-1-[(N,N-dimethyl)aminosulfonyl]-2-butyloxycarbonyl}-Lys -OH (5.11 g, 9.79 mmol, 0.1 M final concentration) and N-hydroxysuccinimide (1.46 g, 12.7 mmol) in 98 mL of CH₂Cl₂. The reaction suspension was stirred at ambient temperature and monitored by C18 HPLC (ELSD). After 2.5 h, the reaction mixture was filtered, and the filtrate was loaded onto a SiliaSep 120 g column. Product was eluted with a step-wise gradient of acetone in hexane (0%, 20%, 30%, 40%, 50%, 60%, 240 mL each). Clean product-containing fractions were combined and concentrated to provide the title compound (4.95 g, 7.99 mmol, 81.6% yield) as a white foam. C18 HPLC, purity was determined by ELSD: 99.7% (RV =10.23 mL). LC-MS (m/z): calc, 520.2; obsd, 520.2 [M+H−Boc]⁺.

(N^{α}-Boc-N^{ε}-{4-Azido-3,3-dimethyl-1-[(N,N-dimethyl)aminosulfonyl]-2-butyloxycarbonyl}-Lys)₄-PEG_{20kDa}. PEG_{20kDa}-(NH)₄ (20.08 g, 0.9996 mmol, 3.998 mmol NH₂, 0.02 M NH₂ final concentration) was dissolved in 145 mL of MeCN. A solution of N^{α}-Boc-N^{ε}-{4-Azido-3,3-dimethyl-1-[(N,N-dimethyl)aminosulfonyl]-2-butyloxycarbonyl}-Lys-OSu (2.976 g, 4.798 mmol) in 50 mL of MeCN was added. The reaction was stirred at ambient temperature and analyzed by C18 HPLC (ELSD). The starting material was converted to a single product peak via three slower eluting intermediate peaks. After 1 h, Ac₂O (0.37 mL, 4.0 mmol) was added. The reaction mixture was stirred 30 min more then concentrated to ˜50 mL by rotary evaporation. The reaction concentrate was added to 400 mL of stirred MTBE. The mixture was stirred at ambient temperature for 30 min then decanted. MTBE (400 mL) was added to the wet solid, and the suspension was stirred for 5 min and decanted. The solid was transferred to a vacuum filter, and washed/triturated with 3×100 mL of MTBE. After drying on the filter for 10 min, the solid was transferred to a tared 250 mL HDPE packaging bottle. Residual volatiles were removed under high vacuum until the weight stabilized to provide the title compound (21.23 g, 0.9602 mmol, 96.1% yield) as a white solid. C18 HPLC, purity was determined by ELSD: 89.1% (RV=10.38 mL) with a 10.6% impurity (RV=10.08).

N^{ε}-{4-Azido-3,3-dimethyl-1-[(N,N-dimethyl)aminosulfonyl]-2-butyloxycarbonyl}-Lys)₄-PEG_{20kDa}. N^{ε}-{4-Azido-3,3-dimethyl-1-[(N,N-dimethyl)aminosulfonyl]-2-butyloxycarbonyl}-Lys)₄-PEG_{20kD a} (19.00 g, 0.8594 mmol, 3.438 mmol Boc, 0.02 M Boc final concentration) was dissolved in 86 mL of 1,4-dioxane. After stirring for 5 min to fully dissolve the PEG, 4 M HCl in dioxane (86 mL, 344 mmol HCl) was added. The reaction was stirred at ambient temperature and analyzed by C18 HPLC (ELSD). The starting material was converted to a single product peak via three faster eluting intermediate peaks. After 2 h, the reaction mixture was concentrated to ˜40 mL. THF (10 mL) was added to the concentrate, and the solution was again concentrated to ˜40 mL. The viscous oil was poured into 400 mL of stirred Et₂O. After stirring at ambient temperature for 20 min, the supernatant was decanted from the precipitate. The wet solid was transferred to a vacuum filter with the aid of 200 mL Et₂O and washed with Et₂O (3×75 mL). The solid was dried on the filter for 10 min then transferred to a tared 250 mL HDPE packaging bottle. Residual volatiles were removed under high vacuum overnight to provide the title compound (17.52 g, 0.8019 mmol, 93.3% yield @4 HCl) as a white solid. C18 HPLC, purity was determined by ELSD: 99.2% (RV=9.34 mL).

### Prepolymer B Wherein C′=Cyclooctynyl

A 4-mL, screw top vial was charged with PEG_{20kDa}-[NH₂]₄ (SunBright PTE-200PA; 150 mg, 7.6 μmol PEG, 30.2 μmol NH₂, 1.0 equiv, 20 mM final amine concentration), MeCN (1.5 mL), and iPr₂NEt (7 μL, 40 1.3 equiv, 27 mM final concentration). A solution of the activated ester cyclooctyne (39 μmol, 1.3 equiv, 27 mM final concentration) was added and the reaction mixture was stirred at ambient temperature. Reactions were monitored by C18 HPLC (20-80%B over 11 min) by ELSD. When complete, Ac₂O (3 μL, 30 μmol, 1 equiv per starting NH₂) was added to the reaction mixture and the mixture was stirred for 30 min. The reaction mixture was then concentrated to a thick oil and suspended in MTBE (20 mL). The resulting suspension as vigorously stirred for 10 min. The resulting solids were triturated three times with MTBE (20 mL) by vigorously mixing, pelleting in a centrifuge (2800 rpm, 4° C., 10 min), and removal of the supernatant by pipette. The resulting solids were dried under vacuum at ambient temperature for no more than 30 min. Stock solutions were prepared in 20 mM NaOAc (pH 5) with a target amine concentration of 20 mM. Cyclooctyne concentration was then verified by treatment with PEG₇-N₃ (2 equiv) and back-titration of the unreacted PEG₇-N₃ with DBCO—CO₂H. Macromonomers prepared using this procedure include those wherein the cyclooctyne group is MFCO, 5-hydroxycyclooctyne, 3-hydroxycyclooctyne, BCN, DIBO, 3-(carboxymethoxy)-cyclooctyne, and 3-(2-hydroxyethoxy)cyclooctyne, prepared using MFCO pentafluorophenyl ester, 5-((4-nitrophenoxy-carbonyl)oxy)cyclooctyne, 3-(4-nitrophenoxycarbonyl)oxycyclooctyne, BCN hydroxysuccinimidyl carbonate, DIBO 4-nitrophenyl carbonate, 3-(carboxymethoxy)cyclooctyne succinimidyl ester, and 3-(hydroxyethoxy)cyclooctyne 4-nitrophenyl carbonate, respectively.

### Example 1. Preparation of a PEEK Membrane Tube

PEEK tubing with 1/16″ outside diameter used for HPLC applications was laser-drilled at Potomac Photonics (Baltimore, MD). The pattern used for drilling is shown in FIG. 1. A total of 1000 10-μm pores were laser drilled 200 μm apart in 5 rows of 200 pores each, arranged parallel to the center axis of the tube, and spaced radially 1 mm apart around the tube circumference.

### Example 2. Microdroplet Emulsification

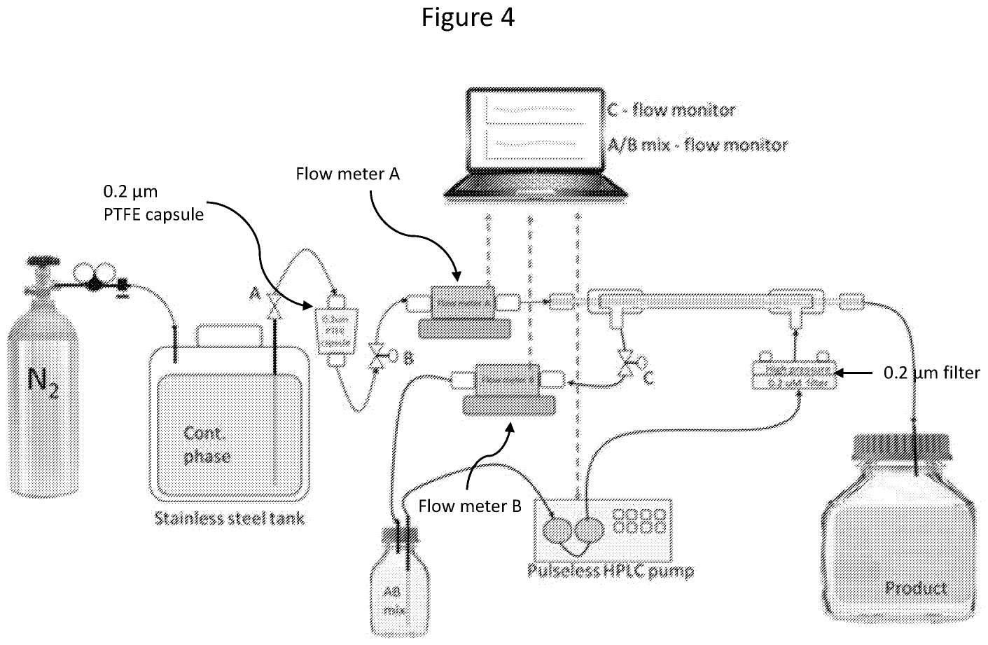

A system for formation of a microdroplet emulsion of a first liquid in a second liquid, in accordance with some embodiments, is illustrated in FIG. 4. A tank of compressed nitrogen fitted with a regulator was used to pressurize a stainless steel tank (Alloy Products) containing the second liquid (continuous phase) to 60 psi. The continuous phase is delivered from the tank by a dip tube, fitted with a ball valve (FIG. 4, component A) to turn flow on or off. Fluorinated ethylene propylene (FEP) tubing (1/8″ OD, 1/16″ ID) is used to carry the continuous phase to a 0.2 μm poly(tetrafluroethylene) (PTFE) membrane capsule filter (Saint Gobain, JKPF0201N1N-NO), then to a needle valve (FIG. 4, component B) used to limit the flow rate, then to a flow meter (Sensirion, SLQ-QT500) for measuring the flow rate, and finally to the inner membrane tube. The first liquid (labeled “AB mix”) is delivered from a GL45 laboratory bottle by a dual piston pulse-dampened HPLC pump (Cole Parmer, Masterflex EW-74931-30), to a 0.2 μm hydrophilic polyvinylidene difluoride (PVDF) membrane filter (Millipore, 47 mm disk, GVWP04700) in a stainless steel high pressure filter housing (Millipore, XX4404700), then onto the membrane tube holder (outer chamber). The membrane tube holder has an exit or “bypass” flow path for the prepolymer solution with a needle valve (FIG. 4, component C) for control of the amount of bypass flow and a flow meter (Sensirion, SLI-2000) for monitoring the bypass flow rate. The flow rate for the first liquid converted to emulsion=(HPLC pump flow rate)−(bypass flow rate). The bypass flow was intended to eliminate a dead end in the interspersion tube holder where polymerized hydrogel may accumulate over time. The maximum operating pressure for the HPLC pump was set to 300 psi. A computer was used to monitor the flow rates from the flow meters and control the HPLC pump. The flow sensors can simply be monitored with the computer application provided with them and the HPLC pump can be manually controlled, or computer-controlled feedback loops may be used to precisely control the flow rates as described in PCT Publication No. WO 2019/152672.

The outer chamber has a piece of type 316 stainless steel tubing having an outside diameter of 1/8″ and an inside diameter of 0.085″. The inner membrane tube is held coaxially inside the outer chamber.

### Example 3. Preparation of Microdroplet Emulsions

A method of preparing microdroplet emulsions using the system of Example 2, in accordance with some embodiments, is illustrated below. An aqueous solution comprising a mixture of two polyethylene glycol “prepolymers” was used as the first liquid, and n-decane containing 1% w/v each of the surfactants Abil® EM90 (cetyl PEG/PPG-10/1 dimethicone, Evonik Industries) and polyglycerol polyricinoleate (PGPR) was used as the second liquid. Preparation of the two prepolymers is disclosed in PCT Publication No. WO 2020/206358, and provided in Preparation A above. Aqueous solutions of prepolymer A (197.11 g, 25 mM azide end group) and prepolymer B (197.11 g, 25 mM cyclooctyne end group) in acetate buffer (38 mM acetate, pH 5.0) were mixed inside of a 500 mL glass GL45 bottle to give the first liquid (“AB mix”). Using the system described in the FIG. 4, a dual piston HPLC style pump with pulse damper was used to feed the first liquid through the pores of the membrane tube of Example 1 (0.0625″ OD, 0.020″ ID, with 1000×10 um, pores) from the outside in at a rate of 10 mL/min. A 0.2 um PES membrane filter in a high pressure stainless steel housing was used between the pump and membrane tube to filter the first liquid. The continuous phase was delivered to the inside of the membrane tube, at a rate of 23 mL/min, from a dip tube within the pressurized (60 psi, N₂) stainless steel tank. A 0.2 um PTFE membrane capsule filter in a polypropylene housing was used between the tank and membrane tube to filter the second liquid. A needle valve downstream of the filter was used to control the flow rate. Flow meters were used to monitor the flow rates of the second liquid (23 mL/min) and the first liquid “bypass” (Note: no bypass flow was used here). The microdroplet emulsion so produced was collected in a glass 2-L GL45 bottle.

### Example 4. Preparation of Microsphere Suspension

The microdroplet suspension of Example 3 was allowed to polymerize to form a suspension of microspheres. The bottle containing the microdroplet emulsion was heated to 40° C. for 18 hours to drive the crosslinking reaction and convert the microdroplet emulsion into a suspension of hydrogel microparticles. Heating was achieved using a silicon band heater fixed to the outside of the jar, with a PID controller and a stainless steel sheathed type K thermocouple immersed in the emulsion for temperature monitoring. Following polymerization the suspension of microspheres was transferred to a washer reactor (disclosed in PCT Publication No. WO 2020/206358) using a 3/16″ inside diameter, 1/4″ outside diameter FEP dip tube. The suspension was stirred at 50-100 rpm and drained into a second washer/reactor at 10 psi through a large-pore sieve (50×250 0.0055″×0.0045″ Dutch weave). The first washer reactor was rinsed with 3×400 mL of the second liquid that was drained into the second washer reactor. The excess second liquid was then drained from the suspension in second washer reactor at 10 psi while stirring at 100-200 rpm; 1200 mL was collected. Water (200 mL) was added to washer/reactor to swell the hydrogel microspheres then the suspension was washed as follows: 6 times with 0.8 kg of heptane, 6 times with 1 L of 190 proof ethanol, and 8 times with 1 kg of 100 mM pH 4 acetate buffer. After the acetate buffer washes, the microsphere suspension (1600 mL) was collected into a 2L GL45 bottle and stored at 4° C. As shown in Table 1, the physical and chemical properties of these microspheres were the same as those prepared using the microfluidics method (disclosed in PCT Publication No. WO 2020/206358). The throughput of the membrane method was significantly higher than that for the microfluidic method, however, with a single membrane tube producing 800 mL/h of microspheres compared with 40 mL/h for a 5-chip microfluidic system having 7 channels per chip. Further, the cost of manufacturing a single membrane tube ($100) is less than the cost of the 5 microfluidic chips ($3500). Whereas the microfluidics chip have a short use life (˜1 L of microsphere suspension for the set of 5 chips) due to erosion of the hydrophobic surface coating, the membrane tube has been reused for >3 L with no apparent degradation in performance.

TABLE 1

| | Microfluidic Chip System | |
|---|---|---|
| Property | (5 chips × 7 channels/chip) | Membrane System (1 tube) |
| particle size (mm) | 60 ± 6 | 62 ± 15 |
| injection force, 27 g needle (kg) | 1.5 ± 0.2 | 1.6 ± 0.2 |
| dissolution t_{RG} (hours) | 9.9 ± 1.0 | 9.8 ± 0.1 |
| [amine]/[PEG] (nmol/mg) | 232 ± 10 | 232 ± 11 |
| slurry pH | 4.05 | 4.11 |
| residual PGPR (ppm) | <1.0 | <1.0 |
| bioburden (CFU/mL) | <10 | <10 |

### Example 5. An Alternative System for Preparing Microsphere Suspension

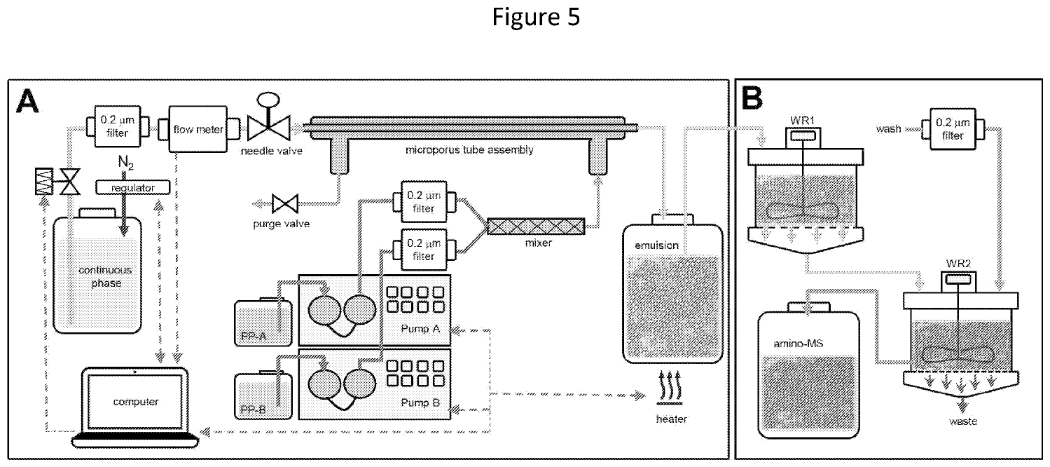

A system for formation of microsphere suspension, in accordance with some embodiments, is illustrated in FIG. 5. The continuous phase composed of decane and surfactants was delivered at a rate of −20 mL/min through a 0.2 μm PTFE membrane filter into the bore of the microporous tube by dip tube transfer from a pressurized tank. The dispersed phase, composed of 3.1 mM each prepolymer A and prepolymer B in pH 5 acetate buffer, was formed by two computer-controlled pulse-dampened HPLC style piston pumps and a static mixer. Alternatively, for prepolymers with slowly-reacting end groups—such as the cyclooctyne-azide end group pair used here—the prepolymer A and B solutions could be pre-mixed in a glass bottle about 5 min prior to delivery, and conveyed to the assembly within a short enough period using a single pump and filter—here, 60 min—to avoid viscosity increases due to premature polymerization of prepolymers. In either case, the prepolymer mixture was pumped at a rate of 10 mL/min through a hydrophilic 0.2 μm PVDF membrane filter into the outer jacket of the assembly. The computer was also used to control the continuous phase flow rate using a PID loop that monitored the flow rate and varied the pressure of the feed tank. Droplets are formed in the flowing continuous phase within the microporous tube. The PEG content of the droplets was two-fold above the aqueous equilibrium swelling concentration of the polymerized hydrogel which allows for a higher rate of volumetric production given that the droplets will swell to twice their volume once exchanged into aqueous media.

After the product emulsion was collected in a 2 L glass jar it was heated to 40° C. for 20 hr to drive the SPAAC crosslinking reaction to completion. The resulting suspension of amino-microspheres (amino-MSs) was then transferred using a dip tube to an assembly consisting of two sequential sieve-bottom washer-reactors (B) that are used to isolate amino-MSs with ˜20-100 μm diameter. Particles over ˜100 μm were removed by the sieve in the first washer-reactor, and desired particles over ˜20 μm and under 100 μm were retained by the sieve in the second washer-reactor. After sieving, the amino-MSs were washed with heptane then ethanol to remove the continuous phase, then exchanged into pH 4.0 AcOH buffer for storage. A typical run produced ˜1500 mL of water-swollen amino-MS slurry of the desired particle diameter in ˜65% to 70% yield based on the prepolymer used.

The critical quality defining attributes for the suspension of amino-MSs in 100 mM AcOH/NaOAc buffer, pH 4.0, are the time to reverse gelation (t_{RG}), the mean particle size and size distribution, chemical identity, the pH of the storage buffer, chemical purity, and the biological purity as measured by the absence of bioburden and endotoxin. A comparison of values for these parameters in amino-MSs produced by the microfluidic process discussed above and the method disclosed herein is given in Table 2. The analytical parameters for amino-MSs produced by both methods agree within acceptable error showing that the method of emulsification has no relevant effect on product quality. The only discernable difference from MSs produced by microfluidics is that the cross-flow process produces amino-MSs with a larger size distribution range, as shown in Table 2. Essentially all of the particles produced have a much smaller diameter than the 210 μm inside diameter of 27 gauge and 159 inside diameter of 30 gauge needles.

TABLE 2

| Assay parameter | Acceptance specs ^{B} | Microfluidic | Cross flow |
|---|---|---|---|
| Dissolution time (t_{RG}, hr) | 9.4- to 10.4 | 9.9 ± 1.0 | 9.8 ± 0.2 |
| Particle size, avg ± SD (μm) | 60 ± 40 | 60 ± 3.4 | 62 ± 15 |
| [nmol Amine]/[mg PEG] | 200- to 250 | 231 ± 15 | 231 ± 11 |
| pH | 3.8- to 4.2 | 4.1 | 4.1 |
| Residual alkyl-azide (μM) | <50 | 27 ± 2.5 | 25 ± 3.0 |
| Residual surfactant PGPR 90 | <50 | <1 ^{C} | <1 ^{C} |
| (PPM) | | | |
| Residual surfactant Abil | <50 | <50 ^{C} | <50 ^{C} |
| EM90 (PPM Si) | | | |
| Bioburden, USP <61> | | | |
| aerobic microbes (CFU/g) | <10 | <10 ^{C} | <10 ^{C} |
| yeast/mold (CFU/g) | <10 | <10 ^{C} | <10 ^{C} |
| Endotoxin, USP<85> | <310 | <0.20 ^{C, D} | 0.24 ± 0.05 ^{D} |
| (EU/mL) | | | |

^{A} Analytical procedures were conducted as previously reported (10).
^{B} Acceptance specs arepreliminary. Reported errors are: range/2 n = 2 for t_{RG}, and SD for all other values.
^{C} Value is lower limit of quantitation.
^{D} A preliminary acceptance specification of <310 EU/ml set based on a dose limit of 5 EU/kg (USP <85>) assuming a 1 ml dose and 62 kg human.

### Example 6. Effect of the Thickness of PEEK Membrane on Droplet Size

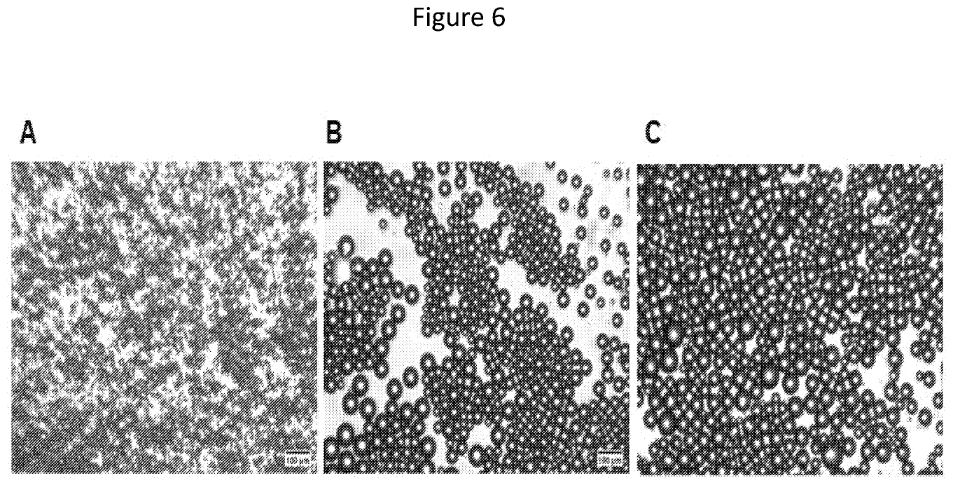

The performance of PEEK membranes of 1/32″ and 1/16″ outside diameter and 0.020″ inside diameter containing 1000 of 10±3 um pores was compared using a continuous phase flow rate of 33 mL/min, and a dispersed phase (water) flow rate of 12 mL/min. The tubes produced emulsified droplets of 1:3±4 μm and 44±14 μm respectively (FIG. 6, A and B). Here, the parameters expected to have the most effect on droplet size: pore size, continuous phase flow rate, and to a lesser extend dispersed phase flow rate were held constant between both tubes, suggesting that the large discrepancy in droplet size is due to the wall thickness of the tubes.

In an attempt to produce larger droplets in the 1/32″ tube the continuous phase flow rate was reduced to 6 mL/min providing droplets of 45±15 μm (FIG. 6, C), however the dispersed phase flow rate had to be reduced to 4 mL/min. This was required to keep the ratio of continuous to dispersed phase high enough to support emulsion stability. Although workable in terms of droplet size the 1/32″ tubes have 3× less volumetric throughput.

In the above description of embodiments, reference is made to the accompanying drawings which form a part hereof, and in which specific embodiments that can be practiced are shown by way of example. Although the disclosed embodiments have been fully described with reference to the accompanying drawings, it is to be noted that various changes and modifications will become apparent to those skilled in the art. Such changes and modifications are to be understood as being included within the scope of the disclosed embodiments as defined by the appended claims. It should be understood that the various embodiments have been presented by way of example only, and not by way of limitation. Likewise, the various diagrams may depict an example architectural or other configuration for the embodiments, which is done to aid in understanding the features and functionality that can be included in the disclosed embodiments. The disclosure is not restricted to the illustrated example architectures or configurations, but can be implemented using a variety of alternative architectures and configurations. Additionally, although the invention is described above in terms of various exemplary embodiments and implementations, it should be understood that the various features and functionality described in one or more of the individual embodiments are not limited in their applicability to the particular embodiment with which they are described. They instead can be applied alone, or in some combination, to one or more of the other embodiments of the invention, whether or not such embodiments are described, and whether or not such features are presented as being a part of a described embodiment. Thus the breadth and scope of the invention should not be limited by any of the above-described exemplary embodiments.

Terms and phrases used in this document, and variations thereof, unless otherwise expressly stated, should be construed as open ended as opposed to limiting. As used herein, the singular forms “a”, “an” and “the” include plural forms, unless the context clearly dictates otherwise. As used herein, and unless otherwise specified, the term “about,” when used in connection with a specified value, is meant to include a value within 5%, within 4%, within 3%, within 2%, within 1%, within 0.5%, or within 0.1% of the specified value.

All references cited are hereby incorporated by reference. The following examples are provided to illustrate and not limit the embodiments of the invention.

1. A membrane comprising a plurality of pores and a surface made from a hydrophobic plastic, wherein the hydrophobic plastic is polyether ether ketone (PEEK).

2. The membrane of claim 1, wherein the membrane is tubular.

3. The membrane of claim 1, wherein the pores have a diameter between about 1 μm and about 100 μm.

4. The membrane of claim 1, wherein the pores are in a patterned arrangement comprising a plurality of rows, wherein each row comprises a plurality of pores.

5. The membrane of claim 4, wherein the distance between centers of two adjacent pores in a row is between about 5-times and about 100-times of the pore diameter.

6. The membrane of claim 4, wherein the distance between two adjacent rows is between about 5-times and about 100-times of the pore diameter.

7. An apparatus comprising the membrane of claim 1, wherein the apparatus further comprises an outer chamber, wherein the membrane is tubular and can be placed inside the outer chamber.

8. The apparatus of claim 7, wherein the outer chamber comprises an inlet and/or an outlet.

9. A system for generating a microdroplet emulsion, comprising the apparatus of claim 7, wherein the system further comprises a means of flowing and pressurizing liquids into the outer chamber of the apparatus or through the tubular membrane, a means of measuring and controlling of the liquid flow rates and pressures, an overall process control device, and/or a means of collecting the microdroplet emulsion generated by the system.

10. The system of claim 9, further comprising one or more sterile filters.

11. A method of generating an emulsion of microdroplets of a first liquid in a second liquid that is immiscible with the first liquid using the apparatus of claim 7, comprising:
flowing the first liquid into the outer chamber; and
flowing the second liquid through the tubular membrane, wherein
the first liquid passes through the pores of the membrane, thereby forming an emulsion of microdroplets of the first liquid in the second liquid.

12. The method of claim 11, wherein the first liquid is an aqueous solution comprising two prepolymers.

13. The method of claim 11, wherein the aqueous solution comprising two multi-arm polyethylene glycols that can react with each other to form a hydrogel.

14. The method of claim 11, further comprising converting the microdroplets to a suspension of microparticles.

15. The method of claim 14, wherein the microparticles comprise crosslinked PEG polymers.

16. The method of claim 15, wherein each crosslink has the formula (I) 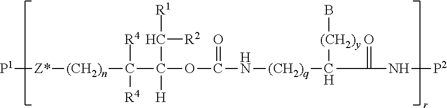
wherein P¹ and P² are each independently r-armed polyethylene glycols, wherein r=2-8;
Z* and B are connecting groups;
n=0-10;
R¹ and R² is each independently H, alkyl, or an electron-withdrawing group with the proviso that at least one of R¹ and R² is an electron-withdrawing group;
each R⁴ is independently C₁-C₃ alkyl, or taken together may form a 3-7 member ring; and
q and y are independently 0-6.

17. A suspension of microparticles prepared by a method of claim 14.
